(19) United States
(12) Patent Application Publication
(10) Pub. No.: US 2003/0018009 A1
(43) Pub. Date: Jan. 23, 2003

(54) Adenosyl-cobalamin fortified compositions

(76) Inventor: Douglas A. Collins, Rochester, MN (US)

Correspondence Address: KING & SPALDING, 191 PEACHTREE STREET, N.E., ATLANTA, GA, 30303-1763, US

(21) Appl. No.: 10/176,138

(22) Filed: Jun. 20, 2002

Related U.S. Application Data
(60) Provisional application No. 60/299,797, filed on Jun. 20, 2001.

(51) Int. Cl. A61K 31/714; A23L 1/30

(52) U.S. Cl. 514/52; 426/72

(57) ABSTRACT

The invention provides food, drink, supplement or other compositions containing a fortifying amount of adenosylcobalamin.

This application claims priority to U.S. provisional application No. 60/299,797 filed on Jun. 20, 2001.

### FIELD OF THE INVENTION

The present invention provides fortified compositions which include a fortifying amount of adenosylcobalamin or hydroxycobalamin, and a food, drink, supplement or other orally ingestible diluent or carrier.

### BACKGROUND OF THE INVENTION

Pernicious anemia, crippling neurological diseases, ataxia and death can result from untreated vitamin B₁₂ deficiency. Cyanocobalamin is a known compound commonly referred to as vitamin B₁₂, however, cyanocobalamin does not occur naturally. Cyanocobalamin (vitamin B₁₂) is produced commercially, and is frequently used as a nutrient for humans because it is a prodrug of the metabolically active vitamin B₁₂ coenzymes. Roth, J. R. et al. (1996) Ann. Rev. Microbiol. 50:137-81. Originally isolated from liver in 1948, vitamin B₁₂ continues to be produced. In 1958, Barker isolated a cofactor of glutamate mutase that was later recognized to be similar to vitamin B₁₂ but missing the cyano group. Weissbach, H. et al. (1960) J. Biol. Chem. 235:1462-1473. This cofactor was determined to be adenosylcobalamin, and despite the discovery of adenosylcobalamin in 1958, cyanocobalamin has been consistently produced commercially and used therapeutically or as a nutritional supplement for more than forty years.

The naturally occurring forms of vitamin B₁₂ found in the body include adenosylcobalamin, methylcobalamin and hydroxycobalamin. Adenosylcobalamin and methylcobalamin are coenzymes for two cobalamin-dependent enzymes—methylmalonyl CoA mutase and methionine synthase. Because the coenzymes are not cyanocobalamin, the coenzymes are not properly designated as vitamin B₁₂. Vitamin B₁₂ has been historically identified as cyanocobalamin. Indeed, Weissbach et al. recognized a relationship between cyanocobalamin and vitamin B₁₂ coezymes. Hogenkamp, H. P. C.; B₁₂: 1948-1998. In: Chemistry and Biochemistry of B₁₂ (R. Banerjee ed.) 1999, Wiley and Sons New York, pp. 1-8. Chemical studies indicated that “unlike the vitamin, the coenzyme lacked the cyanide ion” pp. 3-8 (emphasis added).

Adenosylcobalamin is a necessary cofactor for methyimalonyl CoA mutase. This mutase is involved in proprionate metabolism. Methylcobalamin is required for methionine synthase, which is necessary to recycle the folate cofactor 5-methyltetrahydrofolate back to tetrahydrofolate allowing the folate cofactor to continue to participate in the biosynthesis of purines and pyrimidines. Methionine synthase converts homocysteine to methionine providing methyl groups needed in the methylation cycle and in the synthesis of structures such as myelin. Scott, J. M. (1997) European J. Clinical Nutrition 51, Suppl. I, S49-S53. Because adenosylcobalamin functions in the mitochondria, this coenzyme is intimately connected to energy production and metabolism in general and can play critical roles in the development of obesity.

The chemical structure of adenosylcobalamin is shown in FIG. 1. The fundamental ring system without cobalt (Co) or side chains is called corrin and the octadehydrocorrin is called corrole. The corrin ring has attached seven amidoalkyl (H₂NC(O)Alk) substituents, at the 2, 3, 7, 8, 13, 18 and 23 positions, which can be designated a-g respectively. See D. L. Anton et al., J. Amer. Chem. Soc., 102, 2215 (1980). The 2, 3, 7, 8, and 13 positions are shown in FIG. 1 as positions a-e, respectively. Adenosylcobalamin can be interconverted into hydroxo- or methylcobalamin depending upon cellular demand. A. E. Finkler et al., Arch. Biochem. Biophys., 120, 79 (1967); C. Hall et al., J. Cell Physiol., 133, 187 (1987); M. E. Rappazzo et al., J. Clin. Invest., 51, 1915 (1972) and R. Soda et al., Blood, 65, 795 (1985). Adenosylcobalamin is a known compound and can be chemically synthesized using conventional techniques. Walker T. E. et al. (1974) Biochemistry 13:2650-5.

Adenosylcobalamin, methylcobalamin and hydroxycobalamin are present in minute amounts in animal based foods but not in vegetables. Scott, J. M. (1997) European J. Clinical Nutrition 51, Suppl. I, S49-S53. Hydroxycobalamin forms when adenosylcobalamin, methylcobalamin or substituted cobalamins are exposed to light. Many animals, including humans, require adenosylcobalamin, but do not synthesize it. Bacteria are the primary source of naturally occurring cobalamin. Commercially produced cyanocobalamin is poorly absorbed through the stomach and is generally administered as a sublingual or in injection form.

Three proteins are involved in binding to vitamin B₁₂ and facilitating its absorption. Pepsin and stomach acidity act to release vitamin B₁₂ from these proteins. A protein in saliva, haptocorrin, binds tightly to vitamin B₁₂ at low pH and may protect the molecule from acid hydrolysis and intestinal fauna. Once in the intestine, pancreatic enzymes release vitamin B₁₂ from haptocorrin. Intrinsic factor then binds to the vitamin B₁₂ until the complex reaches the ileum. Cyano-, adenosyl-, hydroxo- and methylcobalamin bind to intrinsic factor with similar affinities. Cyanocobalamin is not readily absorbed directly from the intestine, however, in part because it is not as biologically active.

The intrinsic factor-cobalamin complex binds to specific receptors on the lumenal surface of the intestine and is endocytosed. Intrinsic factor is cleaved intracellularly by intracellular proteases, and the free vitamin B₁₂ binds transcobalamin II and is released into circulation. Adenosylcobalamin in serum is primarily bound to transcobalamin II, and somatic cells take up vitamin B₁₂ bound to transcobalamin II through transcobalamin II receptor mediated endocytosis.

Cobalamin is present in plasma as methylcobalamin, adenosylcobalamin and hydroxocobalamin bound to the specific proteins transcobalamins I and II. Transcobalamin I is a storage form and mainly binds methylcobalamin, whereas mentioned above transcobalamin II is the physiologic B₁₂ transport protein. Cobalamin coenzyme plasma concentration is normally 200 to 750 pg/mL (150 to 550 pmol/L), which represents only about 0.1% of the total body content of coenzymes, most of which is in the liver. Excretion is mainly through the bile and to a lesser extent through the kidneys. The total daily loss is 2 to 15 μg.

The recommended daily allowance of vitamin B₁₂ is 2 μg for adults, 2.2 μg for pregnant women, and 2.6 μg for nursing mothers. Because cyanocobalamin (vitamin B₁₂) is poorly absorbed by itself, nutritional supplements generally contain 50 μg to 2 mg of cyanocobalamin. Adenosylcobalamin and methylcobalamin can be stored in the liver and kidneys for long periods of time and any excess is simply excreted. Problems absorbing vitamin B₁₂ from food can also stem from any disruption in the metabolism of vitamin B₁₂. Genetic abnormalities in the vitamin B₁₂ binding proteins and other vitamin B₁₂ related proteins can result in decreased absorption of vitamin B₁₂. People who are at risk from cobalamin deficiency include those with a gastrointestinal predisposition (e.g., atrophic body gastritis or previous partial gastrectomy), autoimmune disorders [type 1 (insulin-dependent) diabetes mellitus and thyroid disorders], those receiving long term therapy with gastric acid inhibitors or biguanides, and those undergoing nitrous oxide anaesthesia.

Food cobalamin malabsorption is identified by low or low-normal serum cobalamin levels with symptoms of cobalamin deficiency such as mild homocystinuria or methylmalonic aciduria and changes in mental status. The elderly are particularly susceptible to cobalamin deficiency because normal age-related breakdown of the digestive process impairs cobalamin release in the digestive tract. For example, reduced secretion of pancreatic enzymes and hypochlorhydrosis are common age-related factors that contribute to food cobalamin malabsorption. In addition, there is a decreased production of intrinsic factor, which in turn, decreases the absorption of cobalamin. Large doses of oral cyanocobalamin often override such deficiencies.

Cyanocobalamin has historically been used to treat several disorders. Because of the poor absorption of cyanocobalamin through the digestive tract, cyanocobalamin therapy is generally in the form of an injection or sublingual.

Bricker Labs produces a liquid nutritional supplement called B₁₂ Blast that contains 1 mg of cyanocobalamin and 400 μg of folic acid in purified water, fructose syrup, natural raspberry flavor, citric acid, and 0.1% sodium benzoate added as a stabilizer. Importantly, this supplement does not contain adenosyl-cobalamin.

Daily administration of cyanocobalamin in combination with aspirin, antioxidants and niacin is disclosed in U.S. Pat. No. 6,121,249 as a treatment for reducing the severity of atherosclerosis, atherosclerotic central nervous system disease, claudication, coronary artery disease, homocystine related disorders, hypertension, peripheral vascular disease, presenile dementia and/or restenosis in humans.

U.S. Pat. No. 6,110,472 discloses the use of vitamin B₁₂ as a method for treating excessive scalp exfoliation or scalp hyperkeratinization.

U.S. Pat. Nos. 6,093,425 and 6,030,650 disclose nutritional milk formulations containing vitamin B₁₂.

U.S. Pat. Nos. 5,578,336 and 5,569,477 disclose chewing gums containing vitamin B₁₂.

U.S. Pat. No. 6,039,978 disclose dietary foods enhanced with vitamin B₁₂.

U.S. Pat. No. 6,022,853 discloses methods and compositions that include a morphogen in combination with vitamin B₁₂ which, when provided to an individual as a food formulation or supplement, is capable of enhancing tissue development and viability in the individual.

U.S. Pat. No. 5,985,339 discloses refrigeration-shelf-stable ready-to-drink complete nutritional products such as nutritional supplements containing vitamin B₁₂.

U.S. Pat. No. 5,955,321 discloses a process for the preparation of a composition comprising natural vitamin B₁₂ obtained from microbial cells.

U.S. Pat. No. 5,948,443 discloses methods of providing micronutrient and acetylsalicylic acid supplementation needed for both the treatment of nutritional losses and deficiencies and the reduction of the risk of coronary heart disease by administering a daily amount of multivitamins including vitamin B₁₂, minerals, and acetylsalicylic acid.

U.S. Pat. No. 5,925,625 discloses a method for the intranasal administration of a pharmaceutical composition containing a hydroxocobalamin compound to treat cluster headaches. A concentrated dose of hydroxocobalamin is claimed to increase its uptake in the nasal mucosa.

U.S. Pat. Nos. 5,925,377 and 5,869,084 disclose multivitamin formulations containing vitamin B₁₂.

U.S. Pat. No. 5,556,644 discloses a method of improving the immunological status of elderly persons by administering individual dosages of a nutritional supplement containing vitamin B₁₂.

U.S. Pat. No. 4,976,960 discloses a food supplement containing an antioxidant and cobalamin.

U.S. Pat. No. 5,964,224 discloses a method of treating amyotrophic laterla sclerosis (ALS) by parenterally administering about 15 mg to about 100 mg per day of methylcobalamin.

Because of the poor absorption of cyanocobalamin and the severe health problems associated with vitamin B₁₂ coenzyme deficiency, there is a strong need in for compositions that can increase the levels of vitamin B₁₂ coenzyme in a host.

Therefore, it is an object of this invention to provide methods and compositions to treat vitamin B₁₂ coenzyme deficiency in a host.

It is another object of this invention to provide methods and compositions to increase the levels of vitamin B₁₂ coenzymes in a host.

It is yet another object of this invention to provide methods and compositions for regulating the metabolism of a host.

### SUMMARY OF THE INVENTION

The present invention provides fortified compositions which include in combination: (i) a fortifying amount of adenosylcobalamin or hydroxycobalamin, and (ii) a food, drink, supplement or other orally ingestible diluent or carrier. The present invention is based on the surprising discovery that adenosylcobalamin or hydroxycobalamin, preferably adenosylcobalamin, is absorbed significantly more efficiently than other forms of cobalamin, i.e., cyanocobalamin. Adenosylcobalamin is a vitamin B₁₂ coenzyme in which the sixth coordination position of the cobalt atom is linked covalently to the 5′-carbon of 5′-deoxyadenosine (FIG. 1). Hydroxycobalamin is a vitamin B₁₂ coenzyme in which the sixth coordination position of the cobalt atom is linked covalently to a hydroxyl. It has been discovered that the oral administration of fortified food compositions containing a fortifying amount of adenosylcobalamin or hydroxycobalamin, preferably adenosylcobalamin, substantially increases levels of cobalamin in a host. Because adenosylcobalamin and hydroxycobalamin are light sensitive, in a preferred embodiment, the present invention is contained in opaque media or packaged or contained in opaque material. In a particular embodiment, the fortifying amount of adenosylcobalamin and hydroxycobalamin is optionally administered in combination with, or bound to, intrinsic factor, transcobalamin I, transcobalamin II or transcobalamin III, and most preferably intrinsic factor and/or transcobalamin II.

In another embodiment, the invention provides a fortified food composition which comprises in combination: (i) a fortifying amount of adenosylcobalamin or hydroxycobalamin, preferably adenosylcobalamin, (ii) an orally ingestible diluent or carrier; and (iii) at least one additional substance selected from the group consisting of a vitamin, mineral, protein, amino acid, carbohydrate, fat, fatty acid, electrolyte, herb, or herbal extract. In a preferred embodiment, the fortified food composition comprises a protein, more specifically, adenosylcobalamin or hydroxycobalamin, preferably adenosylcobalamin, is bound to intrinsic factor, transcobalamin I, transcobalamin II or transcobalamin III, and most preferably intrinsic factor and/or transcaobalamin II, prior to the fortification process.

In another embodiment, a method for increasing vitamin B₁₂ coenzyme levels in a host comprising administering to a host a fortified food composition containing a fortifying amount of adenosylcobalamin or hydroxycobalamin, preferably adenosylcobalamin, optionally in combination with or bound to a protein such as intrinsic factor, transcobalamin I, transcobalamin II or transcobalamin III, and most preferably intrinsic factor and/or transcobalamin II, in combination with a diluent or carrier is provided.

In yet another embodiment, a method of treating a neurological disorder by orally administering adenosylcobalamin or hydroxycobalamin, preferably adenosylcobalamin, to a host is provided. The cobalamin can be optionally administered with or bound to intrinsic factor, transcobalamin I, transcobalamin II or transcobalamin III, and most preferably intrinsic factor and/or transcobalamin II. In a preferred embodiment, the neurological disease is amyotrophic lateral sclerosis. In a more preferred embodiment, the neurological disorder is Alzheimer's Disease. Because of the increased absorption of adenosylcobalamin or hydroxycobalamin, lower doses can be used than those compared to methylcobalamin or cyanocobalamin. In an alternate embodiment, the neurological disease is multiple sclerosis.

In still another embodiment of the present invention, a multivitamin formulation is provided containing adenosylcobalamin or hydroxycobalamin, preferably adenosylcobalamin optionally mixed with or bound to intrinsic factor, transcobalamin I, transcobalamin II or transcobalamin III, and most preferably intrinsic factor and or transcobalamin II. In preferred embodiments, the multivitamin formulation contains between 0.1 to 2 mg of adenosylcobalamin or hydroxycobalamin, preferably adenosylcobalamin, optionally mixed with or bound to intrinsic factor, transcobalamin I, transcobalamin II or transcobalamin III, and most preferably intrinsic factor and/or transcobalamin II, per dose.

### BRIEF DESCRIPTION OF THE FIGURES

FIG. 1 is a diagram of the chemical structure of adenosylcobalamin.

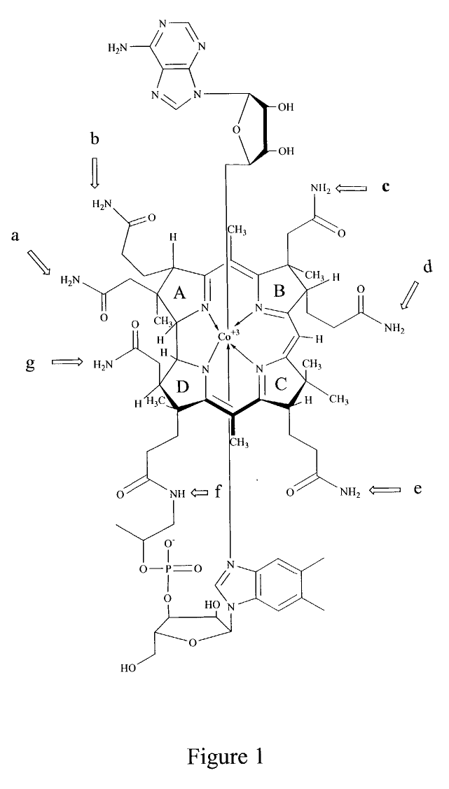

### DETAILED DESCRIPTION OF THE INVENTION

The fortified compositions according to the invention include as an essential component a fortifying amount of adenosylcobalamin or hydroxy-cobalamin, preferably adenosylcobalamin, optionally mixed with or bound to intrinsic factor, transcobalamin I, transcobalamin II or transcobalamin III, and most preferably intrinsic factor and/or transcobalamin II. A fortifying amount of adenosylcobalamin or hydroxycobalamin, preferably adenosylcobalamin, and even more preferably adenosylcobalamin mixed with or bound to intrinsic factor or transcobalamin II, is any amount in excess of naturally occurring cobalamin or in the food composition. Preferred amounts of total added adenosylcobalamin or hydroxycobalamin, preferably adenosylcobalamin optionally mixed with or bound to intrinsic factor, transcobalamin I, transcobalamin II or transcobalamin III, and most preferably intrinsic factor and/or transcobalamin II are between 0.1 μg to 2 mg, and more preferably, between 0.5 or 1 μg to 1 mg, per serving of the fortified food composition. It is understood that the fortified food compositions of the present invention contain 0.1 μg to 2 mg adenosylcobalamin or hydroxycobalamin, preferably adenosylcobalamin optionally mixed with or bound to intrinsic factor, transcobalamin I, transcobalamin II or transcobalamin III, and most preferably intrinsic factor and/or transcobalamin II. An orally ingestible diluent or carrier may for example include a substance selected from a manufactured cereal, fruit or vegetable product, a beverage or beverage concentrate, or any inert diluent, carrier or excipient known in the pharmaceutical or food or beverage art. It is intended generally that adenosylcobalamin or hydroxycobalamin, preferably adenosylcobalamin, may be used in fortified food compositions, in any of the food forms known and practiced in the art. In one embodiment, the fortified food composition is a beverage. In another embodiment, the fortified food composition is a food.

In one embodiment, the fortified food composition is a fortified sports drink. By sports drink, it is meant a beverage consumed to rehydrate and or replenish nutrients and energy after physical activity. Additionally, the fortified sports drink can be consumed in preparation of physical activity. The fortified food composition of the present invention can be in the form of a sports drink containing a fortifying amount of adenosylcobalamin or hydroxycobalamin, preferably adenosylcobalamin, optionally mixed with or bound to a carrier protein, and in a nonlimiting example can include effective amounts of agents effective against muscle cramps together with balanced amounts of carbohydrates and electrolytes. In addition, the fortified sports drink can include ingredients to produce an acid pH. Additives to the fortified sports drink can include fruit flavor, a preservative and carbonation.

The fortified sports drink can be manufactured and sold as a single strength beverage for direct consumption. Alternatively, the fortified sports drink can be in the form of an aqueous concentrate or syrup to be diluted with water to yield a fortified sports drink of desired concentration and taste. The fortified sports drink can also be in dry form, such as a powder or a tablet, which is dissolved in water to yield the fortified food composition of this invention.

The fortified sports drink can be a lightly carbonated beverage supplementing the dietetic requirements of sugar and essential salts in the human body which have been depleted through vigorous physical activity. The fortified drink of this invention can enhance the available energy stores and electrolytes within the body.

The fortified food compositions of the present invention further include any vitamin in addition to adenosylcobalamin or hydroxycobalamin, preferably adenosylcobalamin, optionally bound to or mixed with a carrier protein. For example, the present fortified food compositions which can be in the form of aqueous solutions may include at least one water-soluble vitamin selected from thiamin, niacin, riboflavin, pyridoxine, pantothenic acid, biotin, folic acid and ascorbic acid. Alternatively or additionally, the present fortified food compositions may include at least one oil-soluble vitamin selected from retinol, calciferol, menadione and tocopherol. It is understood that provitamins of the identified vitamins can be used in the present invention. A provitamin is form of a vitamin that is converted into a biological active form of the vitamin in a host. The fortified food compositions of the present invention may also include a desired mineral, including one selected from sodium, potassium, calcium, magnesium, phosphorus, chlorine and sulfur, and additionally or alternatively, at least one element selected from iron, copper, manganese, iodine, cobalt, zinc, molybdenum, fluorine, selenium and chromium.

The fortified food compositions of the present invention can contain an unsaturated fatty acids, for example, lecithin, choline, inositol, linoleic acid, gammalinolenic acid, dihomo-gamma-linolenic acid arachidonic and eicosapentaenoic acids, known to be metabolized in the body to prostaglandins, as well as physiologically compatible derivatives thereof, such as salts, esters and amides of such acids.

The fortified food compositions of the present invention can contain added proteins, such as those derived from gelatin, soy or whey. In an alternate embodiment, the adenosylcobalamin or hydroxylcobalamin can be bound to a carrier or other protein. Examples of such proteins are more specifically, intrinsic factor, transcobalamin I, transcobalamin II or transcobalamin III, though most preferably intrinsic factor and/or transcobalamin II, as well as physiologically compatible derivatives thereof, such as salts, esters and amides of such proteins.

The fortified food compositions of the present invention can also contain a natural or synthetic amino acid. Nonlimiting examples of amino acids include alanine, arginine, aspartic acid, cystine, glutamic acid, glycine, histidine, hydroxylysine, isoleucine, leucine, lysine, methionine, ornithine, phenylalanine, proline, serine, threonine, tryptophan, tyrosine, valine, ornithine, carmitine, hydroxyproline and taurine.

The fortified food compositions of the present invention can contain an added herb or herbal extract. Nonlimiting examples of herbs or herbal extracts include Alfalfa leaf, Alfalfa seed, Angelica root, Anise Seed, Ashwanganda root, Astragalus root, Bee Pollen, Bee Propolis, Bilberries, Black Cohosh root, Black Walnut hulls, Bladderwack, Bayberry bark, Bistort root, Blessed Thistle, Bloodroot, Blue Cohosh root, Boneset, Buckthorn bark, Buchu leaves, Burdock root, Calendula Flower, Cascara Sagrada, Chamomile flowers, Catnip leaf, Cats Claw bark, Chaparral leaf, Chase Tree berry, Chickweed herb, Cleavers herb, Cloves, Colts Foot leaf, Comfrey leaf, Comfrey root, Corn Silk, Crampbark, Cranesbill root, Cumin seed, Damiana leaf, Dandelion root, Devils Claw, Dill seed, Dill weed, Dong Quai root, Dulse, Echinacea Angustifolia, Echinacea Pupurea, Elder flower, Elecampane root, Ephedra Sheep, Eyebright herb, Eucalyptus leaf, False Unicorn root, Fennel seed, Fenugreek seed, Feverfew herb, Fo-Ti Root, Garlic root, Gentian root, Ginger root, Ginkgo Biloba, Ginseng, Goldenseal root, Gotu Kola herb, Guarana seeds, Grape seed extract, Grapefruit seed, Green Tea, Hawthorne berry, Hops flowers, Horsetail, Horehound, Hydrangea root, Hyssop herb, Juniper Berries, Kava Kava root, Kelp, Kola Nut, Lemon peel, Lemongrass, Licorice root, Lily of the Valley root, Lobelia herb, Lungwort lichen, Maca, Marshmallow root, Milk Thistle seeds, Motherwort herb, Muira Puama, Mullein leaf, Nutmeg, Myrrh Gum, Nettle leaf, Oat Straw, Oat Grain, Olive leaf, Orange Peel, Oregon Grape, Parsley leaf, Parsley root, Passion Flower, Pau D'Arco bark, Peppermint leaves, Plantain herb, Prickly Ash bark, Pygeum bark, Red Clover, Red Raspberry leaf, Red Root, Rhubarb root, Rosemary leaf, Sage leaf, Sarsaparilla root, Saw Palmetto Berry, Schisandrae Berries, Scullcap herb, Senna pods, Stillingia root, Shave Grass herb, Sorrel herb, Shepards Purse, Slippery Elm bark, Solomon's Seal root, Spearmint leaves, St. Johns Wort herb, Stevia leaf, Suma Thyme,leaf Tribulus terestris, Tumeric, Usnea, Uva Ursi leaf, Valerian root, Watercress, White Willow bark, Wild Cherry bark, Wild Yam root, Wood Betony herb, Yarrow flowers, Yellow Dock root, and Yohimbe bark.

In another embodiment the invention is offered packaged in a vessel that protects the material from photolyic or other breakdown, for example, opaque bottles or opaque wrapping.

The fortified food compositions of the present invention can include any appropriate amount of a preservative. For example, the material can contain from about 100 ppm to about 1000 ppm, preferably from about 200 ppm to about 650 ppm, more preferably from about 400 ppm to about 650 ppm, of a preservative selected from the group consisting of sorbic acid, benzoic acid, alkali metal salts thereof, and mixtures thereof. The preservative is preferably selected from the group consisting of sorbic acid, potassium sorbate, sodium sorbate and mixtures thereof. Most preferred is potassium sorbate.

In one embodiment of the present invention, the added adenosylcobalamin or hydroxycobalamin, preferably adenosylcobalamin, is at least 50, 60, 70 or 80% pure (i.e., free of other forms of cobalamin). In a preferred embodiment, the added adenosylcobalamin or hydroxycobalamin, preferably adenosylcobalamin, is a single optical isomer. In yet another embodiment, the added adenosylcobalamin or hydroxycobalamin, preferably adenosylcobalamin, is substantially free of cyanocobalamin. Preferably, cyanocobalamin is less than 20%, more preferably less than 10%, and most preferred less than 5% of the added vitamin B₁₂ coenzyme.

In an alternate embodiment, the adenosylcobalamin is in a mixture with hydroxycobalamin. In another embodiment, optionally mixed with or bound adenosylcobalamin or hydroxycobalamin, preferably adenosylcobalamin, is in mixture with optionally mixed with or bound cyanocobalamin.

In another embodiment, the added adenosylcobalamin or hydroxycobalamin, preferably adenosylcobalamin, can be either synthetic or isolated from microbial cultures. The intrinsic factor, transcobalamin I, transcobalamin II or transcobalamin III can be natural or recombinant. Alternatively, a variant of intrinsic factor, transcobalamin I, transcobalamin II or transcobalamin III is used that retains substantially the same biological activity but varies in specific protein sequence. Preferably, the adenosylcobalamin or hydroxycobalamin is not in the form of a microbial paste. Instead, the adenosylcobalamin or hydroxycobalamin is isolated from the microbial organisms using conventional methods known in the art including but not limited to column chromatography, which can then be optionally mixed with or bound to intrinsic factor, transcobalamin I, transcobalamin II or transcobalamin III, preferably intrinsic factor and/or transcobalamin II prior to fortification.

In yet another embodiment, the fortified food composition is a cereal. Cereals can be in the form of ready-to-eat cereals, cereal bars or granola bars. Alternatively, the cereal can require preparation including cooking.

In another embodiment, the fortified food composition is a snack food. In various embodiments, the snack food can be in the form of ready-to-eat packages or require preparation including cooking. Examples of snack foods include, but are not limited to popcorn, pretzels, nuts, such as peanuts, sunflower nuts and pistachio nuts, potato chips, crackers, fries, candy, pudding and popsicles.

In still another embodiment, the fortified food composition is a gelled confection. In one embodiment, a gelled confection can consist primarily of sugars and a fruit base. The gelled confection can be packaged in sheets or in discrete units.

In another embodiment, the fortified food composition is a chewing confection. In one embodiment, a chewing confection can consist primarily of a gum base, optionally flavored with sugar, natural or artificial flavors or fruit juice. The chewing confection can be packaged in sheets or in discrete units.

The fortified food compositions of the present invention can be in the form of fortified bread products, cakes, donuts and cookies. The fortified food compositions can also be in the form of breakfast foods including but not limited to breakfast bars, waffles and pastries. Additionally, the fortified food compositions of the present invention can be health bars or energy bars. Health bars or energy bars can contain carbohydrates, sugars and other nutrients to replenish depleted body stores or to increase body stores in preparation of physical activity. The fortified food compositions can also be formulated to provide a low calorie dietary supplement. Such fortified low calorie dietary supplements can be used as part of a weight-loss program, or as part of weight-maintenance program.

In another embodiment of the present invention, a method of treating cobalamin deficiency is provided comprising administering to a host having low vitamin B₁₂ coenzyme levels a fortified food composition containing a fortifying amount of adenosylcobalamin or hydroxycobalamin, preferably adenosylcobalamin, optionally mixed with one of the carrier proteins described herein. Nonlimiting examples of hosts who can benefit from this treatment include those with a gastrointestinal predisposition (e.g. atrophic body gastritis or previous partial gastrectomy), an autoimmune disorder [type 1 (insulin-dependent) diabetes mellitus and thyroid disorders], those receiving long term therapy with gastric acid inhibitors or biguanides, and those undergoing nitrous oxide anesthesia. Additionally, individuals suffering from pernicious anemia, ataxia, or cobalamin deficiency related neurological disorders can benefit from this administration of the fortified food compositions of the present invention. Because of the critical role vitamin B₁₂ coenzymes play in metabolism and methylation, patients predisposed to or suffering from Alzheimer's disease can also benefit from the present invention.

Other cobalamin related disorders that can be treated with adenosylcobalamin or hydroxycobalamin, preferably adenosylcobalamin fortified food, drink or supplements include cblF-lysosomal accumulation of free cobalamin; cblC and cblD-combined homocystinuria and methylmalonic aciduria; cblA, cblA′, and cblB-defective adenosylcobalamin synthesis; and cblE and cblG-methylcobalamin deficiency.

In another embodiment, a method for treating a neurological disorder in a host is provided comprising orally administering an effective amount of adenosylcobalamin or hydroxycobalamin, preferably adenosylcobalamin to a host. In one embodiment, the neurological disorder is amyotrophic lateral sclerosis. Amyotrophic lateral sclerosis is a progressive disease affecting upper and lower motor neurons in the brain and the spinal cord. In another embodiment, the neurological disorder is multiple sclerosis. Because of the increased absorption of adenosylcobalamin or hydroxycobalamin compared to cyanoand methylcobalamin, and due to the importance of cobalamin to mylenation, high doses are not required. In preferred embodiments, adenosylcobalamin or hydroxycobalamin, preferably adenosylcobalamin, is administered orally on a daily basis in a dose of 0.1 μg to 10 mg, more preferably in a dose of 1 to 5 mg, and most preferably in a dose of 1 to 2 mg.

In still another embodiment of the present invention, a multivitamin formulation containing adenosylcobalamin or hydroxycobalamin, preferably adenosylcobalamin, is provided. In preferred embodiments, isolated adenosylcobalamin or hydroxycobalamin, preferably adenosylcobalamin, can be from 0.11g to 2 mg per dose. Additional vitamins can include at least one water-soluble vitamin selected from thiamin, niacin, riboflavin, pyridoxine, pantothenic acid, biotin, folic acid and ascorbic acid. Alternatively or additionally, the present multivitamin formulation can include at least one oil-soluble vitamin selected from retinol, calciferol, menadione and tocopherol. In preferred embodiments, the multivitamin formulation also can contain folic acid, preferably between 100 to 400 μg of folic acid. It is understood that provitamins of the identified vitamins can be used in the present invention. The multivitamin formulation of the present invention can also include an added mineral, for example, sodium, potassium, calcium, magnesium, phosphorus, chlorine and sulfur, and additionally or alternatively, at least one element selected from iron, copper, manganese, iodine, cobalt, zinc, molybdenum, fluorine, selenium and chromium. Any desired amount can be used, for example, the additional vitamins, minerals, folic acid and elements can be from 5% to 110% of the Recommended Daily Allowance or multiples of the Recommended Daily Allowance. The multivitamin formulations can be used for prenatal vitamin formulations as well as adult vitamin formulation.

Definitions and Use of Terms

As used herein, “fortifying amount” of an added substance refers to an amount exceeding any naturally occurring amount of that substance found in the material to which the fortifying amount is added.

The term “vitamin B₁₂” refers to cyanocobalamin.

As used herein, the term “vitamin B₁₂ coenzyme” refers to adenosylcobalamin, methylcobalamin or both.

As used herein, the term “host” refers to an animal including humans that utilize vitamin B₁₂ coenzymes.

As used herein, the term “and/or” for example in reference to the phrase “intrinsic factor and/or transcobalamin II” refers to cobalamin compositions bound to intrinsic factor separately, transcobalamin II separately, or a combination of separately bound intrinsic factor and transcobalamin II.

Fortifying Dosage Forms

The present invention provides fortified foods, drinks, supplements and other compositions containing optionally mixed with or bound adenosylcobalamin or hydroxycobalamin, preferably adenosylcobalamin. The cobalamin fortified materials can be administered orally to any host in need thereof, including a mammalian host such as a human. Alternatively, the cobalamin fortified materials can be dissolved in any appropriate liquid, such as water or an emulsifier, and administered by the spraying onto of a food ingredient, for example by spray bottle, destined for consumption after the food ingredient is produced.

The fortified compositions herein are also suitably administered by sustained release systems. The sustained release systems can be tailored for administration according to any one of the proposed administration regimes. Slow or extended-release delivery systems, including any of a number of biopolymers (biological-based systems), systems employing liposomes, and polymeric delivery systems, can be utilized with the compositions described herein to provide a continuous or long term source of therapeutic compound.

Suitable examples of sustained release compositions include semi-permeable polymer matrices in the form of shaped articles, e.g., films, microcapsules or microspheres. Sustained release matrices include, for example, polylactides (U.S. Pat. No. 3,773,919), copolymers of L-glutamic acid and γ-ethyl-L-glutamate (Sidman et al., Biopolymers 22:547-556, 1983), or poly-D-(−)-3-hydroxybutyric acid (EP 133,988). Sustained release compositions also include one or more liposomally entrapped optionally mixed with or bound adenosylcobalamin or hydroxycobalamin, preferably adenosylcobalamin. Such compositions are prepared by methods known per se, e.g., as taught by Epstein et al. Proc. Natl. Acad. Sci. USA 82:3688-3692, 1985. Ordinarily, the liposomes are of the small (200-800 Å) unilamellar type in which the lipid content is greater than about 30 mol % cholesterol, the selected proportion being adjusted for the optimal therapy.

A variety of techniques to produce microparticles have been described in the prior art. For example, United Kingdom Patent Application No. 2,234,896 to Bodmer et al. describes a method of forming microparticles by mixing a solution of the polymer dissolved in an appropriate solvent with a solution of a drug. Microparticle formation is then induced by the addition of a phase inducing agent. European Patent Application 0 330 180 to Hyon et al. describes a process for preparing polylactic acid-type microparticles by adding a solution of a drug and a polymer in a mixed solvent to a phase inducing agent and evaporating the original solvent microparticle formation. Other examples of processes for preparing microparticles by phase separation technique have been described in U.S. Pat. Nos. 4,732,763 to Beck et al. and 4,897,268 to Tice et al. and by Ruiz et al. in the International Journal of Pharmaccutics (1989) 49:69-77 and in Pharmaceutical Research (1990) 9:928-934.

The fortified compositions may be administered in combination with a pharmaceutically acceptable vehicle such as an inert diluent or an assimilable edible carrier. They may be enclosed in hard or soft shell gelatin capsules, compressed into tablets or incorporated directly with the food of the patient's diet. For oral therapeutic administration, the substance may be combined with one or more excipients and used in the form of ingestible tablets, buccal tablets, troches, capsules, elixirs, suspensions, syrups, wafers, and the like. Such compositions and preparations should optionally contain at least 0.1% of the substance. The percentage of the compositions and preparations may, of course, be varied and may conveniently be between about 2 to about 60% of the weight of a given unit dosage form. The amount of substance in such therapeutically useful compositions is such that an effective dosage level will be obtained.

Tablets, troches, pills, capsules and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the substance may be incorporated into sustained-release preparations and devices.

Sublingual tablets are designed to dissolve very rapidly. Examples of such formulations include ergotamine tartrate, isosorbide dinitrate, isoproterenol HCl. The formulation of these tablets contain, in addition to the drug, a limited number of soluble excipients, usually lactose and powdered sucrose, but occasionally dextrose and mannitol. The process of making sublingual tablets involves moistening the blended powder components with an alcohol-water solvent system containing approximately 60% alcohol and 40% water.

In addition to the fortified food compositions, the prototype formulation for sublingual tablets may contain a binder such as povidone or HPMC, diluents such as lactose, mannitol, starch or cellulose, a disintegrant such as pregelatinized or modified starch, lubricants such as magnesium stearate, stearic acid or hydrogenated vegetable oil, a sweetener such as saccharin or sucrose and suitable flavoring and coloring agents.

An effective dosages of the adenosylcobalamin or hydroxy-cobalamin can be used for all of the embodiments described herein. Dosages can be determined routinely by any number of methods, including by comparing the in vitro activity, and in vivo activity in animal models. Methods for the extrapolation of effective dosages in mice, and other animals, to humans are known to the art; for example, see U.S. Pat. No. 4,938,949. The amount of the substance required for use in treatment will vary not only with the nature of the condition being treated and the age and condition of the patient and will be ultimately at the discretion of the attendant physician or clinician.

In general, a suitable dose will be in the range of from about 0.1 μg to 2 mg. The substance is conveniently administered in unit dosage form. For example, the fortified food composition can contain 0.1 μg to 2 mg, conveniently 1 μg to 2 mg, most conveniently, 1 to 2 mg of optionally mixed with or bound adenosylcobalamin or hydroxycobalamin, preferably adenosylcobalamin, per unit dosage form.

The substance may conveniently be presented in a single dose or as divided doses administered at appropriate intervals, for example, as two, three, four or more sub-doses per day.

By the term “effective amount” of a compound as provided herein is meant a nontoxic but sufficient amount of the compound to provide the desired effect. As will be pointed out below, the exact amount required will vary from subject to subject, depending on the species, age, and general condition of the subject, the type and severity of the condition that is being treated, the particular compound used, its mode of administration, and the like. Thus, it is not possible to specify an exact “effective amount.” However, an appropriate effective amount may be determined by one of ordinary skill in the art using only routine experimentation.

### EXAMPLES

The following examples are given for the purpose of illustrating various embodiments of the invention and are not meant to limit the present invention in any fashion.

### Example 1

### Fortified Fruit Juice

In one embodiment of the present invention, the fortified food compositions can contain fruit juice, which can provide flavor and nutrition. In a preferred embodiment, the amount of isolated or bound adenosylcobalamin or hydroxycobalamin, preferably adenosylcobalamin, in the fortified fruit juice is between 0.1 μg to 2 mg per serving. The fruit juice in the fortified food compositions can be any citrus juice, non-citrus juice or mixture thereof, which are known in the art. Examples of such fruit juices include, but are not limited to, non-citrus juices such as apple juice, grape juice, pear juice, nectarine juice, currant juice, raspberry juice, gooseberry juice, blackberry juice, blueberry juice, strawberry juice, custard-apple juice, pomegranate juice, guava juice, kiwi juice, mango juice, papaya juice, watermelon juice, cantaloupe juice, cherry juice, cranberry juice, pineapple juice, peach juice, apricot juice, plum juice and mixtures thereof, and citrus juices such as orange juice, lemon juice, lime juice, grapefruit juice, tangerine juice and mixtures thereof. Other fruit juices, fruit flavored juices, and nonfruit juices such as vegetable or botanical juices, can be used as the juice component of the fortified food compositions of the present invention.

Additionally, other vitamin, mineral, protein, amino acid, carbohydrate, fat, fatty acid, electrolyte, herb or herbal extract can be used.

In a preferred embodiment, adenosylcobalamin or hydroxycobalamin is bound to a protein, more specifically, intrinsic factor, transcobalamin I, transcobalamin II or transcobalamin III. Specifically, adenosylcobalamin or hydroxycobalamin bound to intrinsic factor and/or transcobalamin II can be used to fortify the fruit juice.

### Example 2

### Fortified Beverages

In one embodiment, the fortified food compositions of the present invention can be noncarbonated beverage compositions, and typically will, contain an artificial or natural, caloric or noncaloric, sweetener. Carbohydrate sweeteners are preferred, more preferably mono- and or di-saccharide sugars. In preferred embodiments, the fortified beverages can contain between 0.1 μg to 2 mg of isolated adenosylcobalamin per serving.

In another embodiment, the fortified beverage compositions can contain a carbonating agent.

U.S. Pat. No. 6,126,980 to Smith et al. discloses beverages with increased microbial stability and processes for preparing them. The fortified beverages of the present invention can be prepared using similar methods. Briefly, the fortified beverage compositions of the present invention will typically comprise from about 0.1% to about 20%, more preferably from about 5% to about 15%, sugar solids by weight of the beverage products. Suitable sweetener sugars include maltose, sucrose, glucose, fructose, invert sugars and mixtures thereof. These sugars can be incorporated into the beverage products in solid or liquid form but are typically, and preferably, incorporated as a syrup, more preferably as a concentrated syrup such as high fructose corn syrup. For purposes of preparing the fortified beverage compositions of the present invention, these optional sweeteners can be provided to some extent by other components of the fortified beverage products such as the fruit juice component, optional flavorants, and so forth.

Preferred carbohydrate sweeteners for use in the fortified beverage compositions are sucrose, fructose and mixtures thereof. Fructose can be obtained or provided as liquid fructose, high fructose corn syrup, dry fructose or fructose syrup, but is preferably provided as high fructose corn syrup. High fructose corn syrup (HFCS) is commercially available as HFCS-42, HFCS-55 and HFCS-90, which comprise 42%, 55% and 90%, respectively, by weight of the sugar solids therein as fructose.

Optional artificial or noncaloric sweeteners for use in the fortified beverage compositions include, for example, saccharin, cyclamates, sucrose, acetosulfam, Laspartyl-L-phenylalanine lower alkyl ester sweeteners (e.g., aspartame), L-aspartyl-D-alanine amides disclosed in U.S. Pat. No. 4,411,925 to Brennan et al., L-aspartyl-D-senne amides disclosed in U.S. Pat. No. 4,399,163 to Brennan et al., L-aspartyl-L-1-hydroxymethyl-allaneamide sweeteners disclosed in U.S. Pat. No. 4,338,346 to Brand, L-aspartyl-1-hydroxyethylakaneamide sweeteners disclosed in U.S. Pat. No. 4,423,029 to Rizzi, and L-aspartyl-D-phenylglycine ester and amide sweeteners. A preferred sweetener is aspartame.

The fortified beverage compositions herein can further comprise any other ingredient or ingredients typically used as optional beverage ingredients. Such optional ingredients include flavorants, preservatives, colorants and so forth.

The fortified beverage compositions can further comprise any amount, including from 1 to about 110% of the U.S. Recommended Daily Allowance (RDA) of vitamins and minerals other than adenosylcobalamin or hydroxycobalamin. Nonlimiting examples include vitamin A, including its provitamins such as beta carotene, and ascorbic acid.

Additionally, other vitamin, mineral, protein, amino acid, carbohydrate, fat, fatty acid, electrolyte, herb or herbal extract can be used.

In a preferred embodiment, adenosylcobalamin or hydroxycobalamin used to fortify the beverage is mixed with or bound to a protein, more specifically, intrinsic factor, transcobalamin I, transcobalamin II or transcobalamin III. Specifically, adenosylcobalamin or hydroxycobalamin bound to intrinsic factor and/or transcobalamin II can be used to fortify the beverage.

### Example 3

### Fortified Dairy Products

In yet another embodiment, the fortified food composition may be a dairy based product such as a milk beverage, a confectionery product, ice cream, or yogurt. In preferred embodiments, 0.1 μg to 2 mg per serving of isolated or bound adenosylcobalamin or hydroxycobalamin, preferably adenosyl-cobalamin, is added to the dairy based product.

U.S. Pat. No. 5,820,903 discloses methods for producing yogurt. Briefly, yogurt can be prepared with raw milk, that may contain a combination of whole milk, skim milk, condensed milk, dry milk (dry milk solids non-fat or, equivalently, “MSNF”), grade A whey, cream and/or such other milk fraction ingredients as buttermilk, whey, lactose, lactalbumins, lactoglobulins, or whey modified by partial or complete removal of lactose and/or minerals, other dairy ingredients to increase the nonfat solids content, which are blended to provide the desired fat and solids content. While not preferred, the milk base can include a filled milk component, i.e., a milk ingredient having a portion supplied by a non-milk ingredient, e.g., oil or soybean milk.

Although the present invention is discussed in terms of fermented bovine milk products such as yogurt, one skilled in the art will appreciate that the present invention is also suitable for use in a wide variety of thickened dairy products, particularly fermented dairy products such as kefir, sour cream, butter and the like.

Also, while bovine milk is preferred, other milks can be used in substitution for bovine milk whether in whole or in part, e.g., goat, sheep or equine milk. Milk alternatives can also be used such as soy bean based beverages.

Conveniently, the raw milk and sweeteners (such as fructose, corn syrup, sucrose) can be blended in a mix tank and stored in a milk silo. Stabilizers and thickeners such as starch, gelatin, pectin, agar and carrageenan may also be added if desired. The minor dry ingredients are combined with the sweetened milk to form the milk base conveniently in a separate mixing vessel.

The milk base is then homogenized in a conventional homogenizer to disperse evenly the added materials and the fat component supplied by various ingredients thereby forming an homogenized milk base. If desired, the milk base can be warmed prior to homogenization from typical milk storage temperatures of about 5° C. to temperatures of about 65° to 75° C.

This homogenized milk base is then pasteurized, typically by heating for times and temperatures effective to accomplish pasteurization to form a pasteurized milk base. As is well known, the milk base can be heated to lower temperatures for extended times or alternately to higher temperatures for shorter times. Intermediate temperatures for intermediate times can also be employed. Other pasteurization techniques can be practiced (e.g., light pulse, ultra high pressure, etc.) if effective and economical. In certain commercial practices, the sequence of the homogenization and pasteurization steps can be reversed.

The homogenized and pasteurized base is then brought to incubation temperature. The homogenized and pasteurized milk blend is then inoculated with a desired culture. Usually, a combination of lactobacillus bulgaricus and streptococcus thermophilus bacteria is added to begin the fermentation process. Fermentation is quiescently continued until the pH of the milk blend reaches approximately 4.4 to 4.6 to form the yogurt base. Depending upon temperature and amount of culture added, this may take from about three to about 14 hours. It is important that the mixture not be agitated during the fermentation process to allow proper curd formation. When the proper pH has been reached, the yogurt is cooled to arrest further growth and any further drop in the pH.

The particular fermentation endpoint pH can vary modestly. Typically, the endpoint pH can range from about 4.2 to 4.6, preferably about 4.45 to 4.55.

The person of ordinary skilled in the art will appreciate that the adenosylcobalamin fortification methods herein rely upon post fermentation rather than pre-fermentation addition. In preferred embodiments, optionally mixed with or bound adenosylcobalamin or hydroxylcobalamin, preferably adenosyl-cobalamin, can be added to the yogurt in amounts between 0.1 μg to 2 mg per serving.

Although a live yogurt product is preferred, the present invention can also be used in yogurt-based foods as distinguished from a yogurt product. For example, a shelf stable yogurt-based product is prepared by heat treating a yogurt to inactivate the culture and packaging aseptically. In this variation, the pH of the yogurt based product can be adjusted for taste or for compatibility with other ingredients. For example, the pH can be adjusted upwards substantially for a chocolate flavored yogurt based product.

Additionally, other vitamins, minerals, proteins, amino acids, carbohydrates, fats, fatty acids, electrolytes, herbs or herbal extracts can be used in conjunction with the fortified dairy product.

In a preferred embodiment, adenosylcobalamin or hydroxycobalamin is bound to a protein, more specifically, intrinsic factor, transcobalamin I, transcobalamin II or transcobalamin III prior to fortification of the dairy product. Specifically, adenosylcobalamin or hydroxycobalamin is bound to intrinsic factor and or transcobalamin II to fortify the dairy product.

### Example 4

### Fortified Cereal

In another embodiment, the present invention discloses a fortified food composition consisting of a cereal ingredient containing a fortifying amount of optionally mixed with or bound adenosylcobalamin or hydroxycobalamin. Other products, such as waffles, snack bars, toaster pastries, and pastry products, can be fortified in the same manner with optionally mixed with or bound adenosylcobalamin or hydroxycobalamin, either alone or in combination with an additional vitamins, minerals, proteins, amino acids, carbohydrates, fats, fatty acids, electrolytes, herbs or herbal extracts. Cereal ingredients include plain or puffed wheat, rice, oat, corn, barley, rye, millet, sorghum, amaranth seed and mixtures of the above can also be used in the preparation of the fortified food compositions. In preferred embodiments, 0.1 μg to 2 mg of isolated or bound adenosylcobalamin or hydroxycobalamin is added per serving to the fortified cereal food composition.

In another embodiment, the fortified food composition is a cereal product. U.S. Pat. No. 3,494,769 describes a breakfast cereal suitable for use as cold cereal by the addition of milk, or as a hot cereal by the addition of hot water. The cereal is prepared by heating rolled oats to cook the starch and protein contained therein, applying liquid milk in sufficient quantity only to wet the oats and to distribute it evenly throughout the oat product, and then drying the wet product to crispness, producing a crunchy product. During the manufacturing process, the flaky or granular cereal can be sprayed or sprinkled with liquid milk in which isolated adenosylcobalamin, sugar, salt, fruit juice puree, and/or flavoring materials are dissolved, whereby the mixture is absorbed by the oat flakes and evenly distributed throughout the body of the flakes. Alternatively, isolated or bound adenosylcobalamin or hydroxycobalamin dissolved in any appropriate liquid, such as water or an emulsifier, can be sprayed on the cereal ingredient after the cereal ingredient is produced.

Cream, butterfat or cream substitute may be added to the milk to improve the flavor and the texture of the product. The cream or dry cream substitute may be mixed with the milk or it may be added to the cereal in a conventional mixer after the milk containing the other additives has been added. If a dry cream substitute is used, it may be dusted onto the cereal while the mixer is operating. The amount of milk added to the cereal is determined by the desired crunchiness of the resulting product, i.e., if a relatively small amount of milk is used and little fruit is added, the product will be relatively soft and water absorptive and not crunchy, or if a higher proportion of milk with fruit is used to wet the cereal, which is thereafter dried, it is crunchy.

### Example 5

### Fortified Snack Food

In another embodiment, the present invention discloses a fortified food composition consisting of a snack food containing a fortifying amount of isolated or bound adenosylcobalamin or hydroxycobalamin. Snack foods include, but are not limited to popcorn, pretzels, nuts, such as peanuts, sunflower nuts and pistachio nuts, potato chips, crackers, fries, candy, pudding and popsicles. In preferred embodiments, 0.1 μg to 2 mg of isolated or bound adenosylcobalamin or hydroxycobalamin is added per serving to the fortified cereal food composition.

Artificial or natural flavorings, cream, butterfat or cream substitute may be added to the snack product to improve the flavor and the texture. The artificial or natural flavorings, cream, butterfat, cream substitute or dry cream substitute may be added to the snack product in a conventional mixer after the other additives has been added. If a dry cream substitute is used, it may be dusted onto the snack product while the mixer is operating. The amount of artificial or natural flavorings, cream, butterfat, cream substitute or dry cream substitute added to the snack product is determined by the desired crunchiness and flavor of the resulting product, i.e., if a relatively small amount of milk is used and little fruit is added, the product will be relatively soft and water absorptive and not crunchy, or if a higher proportion of milk with fruit is used to wet the snack product, which is thereafter dried, it is crunchy.

### Example 6

### Fortified Confections

In another embodiment, the present invention provides fortified sweetened confections. In preferred embodiments, 0.1 μg to 2 mg per serving of isolated or bound adenosylcobalamin or hydroxycobalamin is added to the sweetened products.

U.S. Pat. No. 6,077,557 discloses calcium fortified gelled sweetened fruit products. Similar compositions and methods are applicable to isolated or bound adenosylcobalamin or hydroxycobalamin fortified sweetened confections. Briefly, a principal essential component of the present invention food products is one or more nutritive carbohydrate sweeteners or sugars. The present confections essentially comprise about 60% to about 85% of such nutritive carbohydrate sweeteners, preferably about 60% to about 75%, and for best results about 65% to about 70%. Such sugars also influence the texture and structure of the present products.

The term “nutritive carbohydrate sweetening agent” is used herein to mean those typical purified sweetening agents conventionally used in food products. Of course, the present nutritive carbohydrate-sweetening agents are to be distinguished from non-nutritive carbohydrate high potency sweetening agents such as saccharine, cyclamate, and the like. Additionally, the present nutritive carbohydrate-sweetening agents are to be distinguished from such protein-based sweetening agents as aspartame, thaumatin and monellin.

Suitable materials for use as nutritive carbohydrate sweetening agents are well known in the art. Examples of sweetening agents include both monosaccharide and disaccharide sugars such as sucrose, invert sugar, dextrose, lactose, honey, maltose, fructose, maple syrup and corn syrup or corn syrup solids. Preferred nutritive carbohydrate sweetening agents are those selected from the group consisting of sucrose, glucose, fructose, corn syrup solids, and honey. Highly preferred nutritive carbohydrate sweetening agents are those selected from the group consisting of sucrose, corn syrup solids, and fructose. Of course, mixtures of the above-noted materials are contemplated herein.

In a preferred embodiment, the ratio of monosaccharide to disaccharide sweeteners is controlled so as to minimize the development of unwanted properties in the finished food product over storage such as the development of crystals. To that end, the ratio can be and preferably does range from about 0.5:1 to about 1.8:1, and more preferably, about 0.7:1 to about 1.5:1.

In preferred embodiments, the fortified sweetened confections herein are fruit products. In such preferred embodiments, the fortified sweetened confections are further essentially characterized by having at least a portion of the nutritive carbohydrate sweeteners as being provided by or from fruit sources or fruit solids. The fruit solids can be derived from fruit purees prepared from whole fruit flesh or if such purees have been partially dehydrated, fruit paste. The term “puree” has been used in the art to refer to both heat treated, e.g., boiled and untreated food pulp. As used herein, however, “puree” is meant to refer both to heat and unheat-treated whole fruit pieces, which have been mechanically transformed into fluids. Thus, the present comminuted fruit material can be distinguished from discrete individual pieces of intact fruit flesh.

Both unseeded and, preferably, deseeded purees can be used. Fruit puree generally contains about 35 to 90% moisture. Other edible fruit portions, such as fruit pulp can also supply the fruit solids component. Fruit pulp is the material remaining after fruit juices have been removed from fruit puree. Additionally useful herein for supplying the fruit solids are various fruit juices whether single strength or concentrated.

Fruit materials from any fruit can be used herein. Examples of such fruits useful herein include apricot, pineapple, lemon, orange, peach, pear, lime, banana, grape, mango, apple, tomato, blackberry, plum, watermelon, blueberry, raspberry, strawberry, current, cherry, cranberry, and mixtures thereof. Preferred fruits are selected from the group consisting of apples, strawberries, cherries, pears, blueberries, raspberries, grapes, oranges and mixtures thereof. Most highly preferred for use herein are grapes, strawberries, pears, oranges and cherries.

Fresh fruit is preferable and useful for preparing the products herein. However, previously frozen fruit, canned fruit, partially dehydrated fruit or rehydrated fruit, as well as frozen juices, concentrates, nectars, powders or frozen juice pulps are also suitable.

While this embodiment is primarily directed towards fortified fruit containing products, the skilled artisan will appreciate that the present invention is equivalently applicable to all edible plant solids, especially ordinary garden-variety vegetables. The sugars, flavors, acids, pectinaceous or cellulosic fibers and ash of which plant solids are typically comprised are intended to be included within the term edible plant solids. However, “edible plant solids” is not intended to include such starch fractions as wheat or other cereal flours nor oleaginous materials such soybean oil. The present fruit solids can be wholly or partially replaced with equivalent amounts of ordinary garden vegetable solids

Fruit juice solids from fruit sources such as grape juice, apple juice and pear juice are preferred. If present, such juice solids can constitute about 0.1 to about 70% of the finished fortified fruit snack products herein.

In even more preferred embodiments, the present compositions comprise from about 5 to 100% (dry weight basis) of the nutritive carbohydrate sweetener component of fruit or plant solids. Preferably, the fruit solids are present at from about 5 to 25% of the sweetener component. More preferably, the fruit solids include about 5 to 15% of the nutritive carbohydrate sweetener component.

If desired, the present fortified sweetened confections can additionally comprise supplemental high potency sweeteners such as saccharine, aspartame, thaumatin, potassium acetylsulfame and mixtures thereof. Other suitable high potency sweeteners that become permitted for use or commercially available from time to time can also be used.

Suitable gelling agents are known in the art and the skilled artisan will not have difficulty in selecting suitable gelling agent(s) for use herein. Gelling agents are distinguishable from mere thickening agents. Exemplary gelling agents include gelatin, gellan gum, carbohydrate gel forming polymers (such as pectin, gel forming starches, dextran, agar, and mixtures thereof), and mixtures thereof and wherein the gel is free of alginates. Among the gel forming carbohydrate polymer gel forming ingredients, pectin and gel forming starches are preferred. Preferred for use herein is gelatin or pectin.

It will be appreciated that the fruit solids, if employed, will additionally provide some native amount of pectin. Preferably, the total pectin (including both the native pectin associated with the fruit solids and added pure pectin) content ranges from about 0.9% to about 2%.

### Example 7

### Fortified Chewing Gum

In another embodiment, the present invention provides fortified gum products. In preferred embodiments, 0.1 μg to 2 mg per serving of isolated or bound adenosylcobalamin or hydroxycobalamin is added to the gum products.

Optionally, the gum products comprise one or more nutritive carbohydrate sweeteners or sugars. In one non-limiting embodiment, the present gum products essentially comprise about 60% to about 85% of such nutritive carbohydrate sweeteners, preferably about 60% to about 75%, and for best results about 65% to about 70%. Such sugars also influence the texture and structure of the present products.

Suitable materials for use as nutritive carbohydrate sweetening agents are well known in the art. Examples of sweetening agents include both monosaccharide and disaccharide sugars such as sucrose, invert sugar, dextrose, lactose, honey, maltose, fructose, maple syrup and corn syrup or corn syrup solids. Preferred nutritive carbohydrate sweetening agents are those selected from the group consisting of sucrose, glucose, fructose, corn syrup solids, and honey. Highly preferred nutritive carbohydrate sweetening agents are those selected from the group consisting of sucrose, corn syrup solids, and fructose. Of course, mixtures of the above-noted materials are contemplated herein.

In a preferred embodiment, the ratio of monosaccharide to disaccharide sweeteners is controlled so as to minimize the development of unwanted properties in the finished food product over storage such as the development of crystals. To that end, the ratio can be and preferably does range from about 0.5:1 to about 1.8:1, and more preferably, about 0.7:1 to about 1.5:1.

In even more preferred embodiments, the present compositions essentially comprise from about 5 to 100% (dry weight basis) of the nutritive carbohydrate sweetener component of fruit or plant solids. Preferably, the fruit solids are present at from about 5 to 25% of the sweetener component. More preferably, the fruit solids are comprise about 5 to 15% of the nutritive carbohydrate sweetener component.

If desired, the present fortified gum products can additionally comprise supplemental high potency sweeteners such as saccharine, aspartame, thaumatin, potassium acetylsulfame and mixtures thereof. Other suitable high potency sweeteners that become permitted for use or commercially available from time to time can also be used.

Suitable gum bases are known in the art and the skilled artisan will not have difficulty in selecting suitable base(s) for use herein.

The invention has been described with reference to illustrative embodiments and techniques. However, it should be understood that many variations and modifications may be made while remaining within the spirit and scope of the invention.

We claim:

1. A fortified food composition comprising a fortifying amount of adenosylcobalamin, optionally mixed with or bound to intrinsic factor, transcobalamin I, transcobalamin II and/or transcobalamin III.

2. A fortified food composition according to claim 1, wherein the adenosylcobalamin is bound to intrinsic factor or transcobalamin II.

3. A fortified food composition according to claim 1, wherein the food includes a substance selected from the group consisting of a manufactured cereal, a fruit or vegetable product, a beverage or beverage concentrate, a ground meat product or a vegetable analog thereof, and any inert diluent, carrier or excipient known in the pharmaceutical art.

4. A fortified food composition according to claim 1, which comprises at least one additional ingredient selected from the group consisting of thiamin, riboflavin, niacin, pyridoxine, pantothenic acid, biotin, folic acid, or ascorbic acid, retinol, calciferol, tocopherol, or menadione, potassium, calcium, phosphorus, magnesium, chlorine and sulfur, iron, copper, iodine, manganese, cobalt, zinc molybdenum, fluorine, selenium chromium, unsaturated fatty acids, an herb or herbal extract, and folic acid.

5. A method for increasing vitamin B₁₂ coenzyme levels in a host comprising administering to the host a fortified food composition comprising a fortifying amount of adenosylcobalamin, optionally mixed with or bound to intrinsic factor, transcobalamin I, transcobalamin II and/or transcobalamin III.

6. The method of claim 5, wherein the adenosylcobalamin is bound to intrinsic factor and/or transcobalamin II.

7. The fortified food composition of claim 1 or 2, wherein the food composition is a fermented dairy product.

8. The fortified food composition of claim 1 or 2, wherein the food composition is a milk beverage.

9. The fortified food composition of claim 1 or 2, wherein the food composition is a snack food.

10. The fortified food composition of claim 1 or 2, wherein the food composition is a chewing gum.

11. A cereal, comprising:
(i) at least one cereal ingredient, and
(ii) about 0.1 μg to 2 mg of isolated adenosylcobalamin.

12. A method for treating food cobalamin malabsorption in a host comprising orally administering a fortified food composition comprising a fortifying amount of adenosylcobalamin, optionally mixed with or bound to intrinsic factor, transcobalamin I, transcobalamin II and/or transcobalamin III.

13. The method of claim 12, wherein the adenosylcobalamin is bound to intrinsic factor and/or transcobalamin II.

14. The fortified food composition of claim 1 or 2, wherein the food composition is a sweetened confection.

15. The fortified food composition of claim 1 or 2, wherein the food composition is a beverage.

16. The fortified food composition of claim 1 or 2, wherein the food composition is a baked bread product.

17. A method for treating a neurological disorder involving a cobalamin deficiency, comprising administering a fortified food composition comprising a fortifying amount of adenosylcobalamin, optionally mixed with or bound to intrinsic factor, transcobalamin I, transcobalamin II and/or transcobalamin III.

18. The method of claim 17, wherein the adenosylcobalamin is bound to intrinsic factor and/or transcobalamin II.

19. A method for treating pernicious anemia or ataxia comprising administering a fortified food composition comprising a fortifying amount of isolated adenosylcobalamin, optionally mixed with or bound to intrinsic factor, transcobalamin I, transcobalamin II and/or transcobalamin III.

20. The method of claim 19, wherein the adenosylcobalamin is bound to intrinsic factor and/or transcobalamin II.

21. A method of treating cobalamin deficiency in a host who has atrophic body gastritis, comprising administering a fortified food composition comprising a fortifying amount of isolated adenosylcobalamin, optionally mixed with or bound to intrinsic factor, transcobalamin I, transcobalamin II and/or transcobalamin III.

22. The method of claim 21, wherein the adenosylcobalamin is bound to intrinsic factor and/or transcobalamin II.

23. The food composition of claim 1 or 2, wherein the adenosylcobalamin is at least 50% pure.

24. The food composition of claim 1 or 2, wherein the adenosylcobalamin is at least 80% pure.

25. The food composition of claim 1 or 2, wherein the adenosylcobalamin is a single optical isomer.

26. A method of treating cobalamin deficiency in a host who has an autoimmune disorder, comprising administering a fortifying amount of isolated adenosylcobalamin, optionally mixed with or bound to intrinsic factor, transcobalamin I, transcobalamin II and/or transcobalamin III. wherein the adenosylcobalamin.

27. A method of treating cobalamin deficiency in a host who is receiving long term therapy with gastric acid inhibitors or biguanides, comprising administering a fortified food composition comprising a fortifying amount of isolated adenosylcobalamin, optionally mixed with or bound to intrinsic factor, transcobalamin I, transcobalamin II and/or transcobalamin III.

28. The food composition of claim 1 or 2, substantially free of cyanocobalamin.

29. The food composition of claim 1 or 2, substantially free of methylcobalamin.

30. A method for treating neurological disorders in a host, comprising orally administering to a host 0.1 μg to 10 mg of isolated adenosylcobalamin, optionally mixed with or bound to intrinsic factor, transcobalamin I, transcobalamin II and/or transcobalamin III on a daily basis.

31. The method of claim 30, wherein the adenosylcobalamin is bound to intrinsic factor and/or transcobalamin II.

32. The method of claim 30 or 31, wherein the neurological disorder is Alzheimer's disease.

33. The method of claim 30 or 31, wherein the neurological disorder is amyotrophic lateral sclerosis.

34. The method of claim 30 or 31, wherein the neurological disorder is multiple sclerosis.

35. A multivitamin formulation comprising adenosylcobalamin, optionally mixed with or bound to intrinsic factor, transcobalamin I, transcobalamin II and/or transcobalamin III, and at least one other ingredient selected from the group consisting of a vitamin, mineral, protein, amino acid, carbohydrate, fat, fatty acid, electrolyte, herb or herbal extract.

36. The formulation of claim 35, wherein the adenosylcobalamin is bound to intrinsic factor and/or transcobalamin II.

37. A method for increasing adenosylcobalamin levels in a host comprising administering to the host a fortified food composition comprising a fortifying amount of isolated adenosylcobalamin, optionally mixed with or bound to intrinsic factor, transcobalamin I, transcobalamin II and/or transcobalamin III.

38. The method of claim 37, wherein the adenosylcobalamin is bound to intrinsic factor and/or transcobalamin II.
